(19) United States
(12) Patent Application Publication
(10) Pub. No.: US 2022/0211620 A1
(43) Pub. Date: Jul. 7, 2022

(54) Pain Relieving Spray

(71) Applicant: Animal Ethics Pty Ltd, Crestmead (AU)

(72) Inventors: Charles Robert Olsson, Crestmead (AU); Meredith Sheil, Hunters Hill (AU)

(21) Appl. No.: 17/613,800

(22) PCT Filed: May 25, 2020
(86) PCT No.: PCT/AU2020/050517

§ 371 Date: Nov. 23, 2021

(30) Foreign Application Priority Data
May 28, 2019 (AU) 2019901831

(51) Int. Cl. A61K 9/12; A61K 31/137; A61K 31/167; A61K 31/245; A61K 31/445; A61K 45/06; A61K 9/02

(52) U.S. Cl. CPC A61K 9/124; A61K 31/137; A61K 31/167; A61K 9/02; A61K 31/445; A61K 45/06; A61K 31/245

(57) ABSTRACT

A pain relieving aerosol spray (pain relieving and pain preventing aerosol spray) comprising a pain relieving composition and at least one refrigerant, wherein the pain relieving aerosol spray is capable of providing rapid onset local anaesthesia of intact skin and/or a wound of a subject to which it is applied and further pain relief due to the pain relieving composition remaining on the wound of the subject. The pain relieving composition comprises a liquid gel matrix that contains at least one anaesthetic agent, a vasoconstrictor, and an antiseptic agent. The spray can be used for a surgical procedure or animal husbandry procedure, such as castration or branding, or treating a burn or wound.

### CROSS-REFERENCE TO RELATED APPLICATIONS

This application is the United States national phase of International Application No. PCT/AU2020/050517 filed May 25, 2020, and claims priority to Australian Provisional Patent Application No. 2019901831 filed May 28, 2019, the disclosures of which are hereby incorporated by reference in their entirety.

### TECHNICAL FIELD

This invention relates to a pain relieving aerosol spray, its method of manufacture, and to its use in traumatic wound care, surgical procedures or animal husbandry procedures. In particular, the invention concerns a pain relieving aerosol spray comprising a pain relieving composition and a refrigerant for providing rapid onset local anaesthesia with additionally prolonged analgesic effect.

### BACKGROUND ART

Pain from a wound (eg. laceration, surgical incision, ulcer or burn) is initiated by a stimulation of traumatized nerve fibres and is intensified by a local inflammatory response that occurs over ensuing 24-48 hours and results in local tissue swelling and oedema. Pain from an open wound is also further intensified and prolonged by a sensitization reaction of higher nerve function which also occurs over ensuing hours and days, and may lead to lower pain thresholds and prolonged hypersensitivity of surrounding tissues.

Such wounds are usually treated by closing or sealing the wound, such as by suturing, adhesive plastering, bandaging or other practice. Closing the wound stops bleeding, protects traumatised tissues and nerve fibres from dehydration, ongoing environmental exposure, risk of infection and ongoing painful stimulation. Pain therefore abates as the inflammatory response and tissue oedema subsides.

In many cases, there may be a delay in treatment and/or wounds cannot be treated as required and are therefore left open to heal by secondary intention. This applies to a very large number of acute traumatic and/or surgically induced wounds, particularly in animals. Examples include:
Wounds in animals, including traumatic wounds and/or those caused by husbandry procedures such as branding and mulesing.
Wounds in mass human trauma situations, such as earthquakes, floods and wars.
Wounds that occur in remote locations and Third World countries where medical attention may be limited or unavailable.

The inventors' earlier patents U.S. Pat. Nos. 8,822,416, 8,960,128 and 9,592,318 describe topical analgesic and anaesthetic compositions, which in one commercial form is marketed under the trade mark Tri-solfen™. Although the commercial form is used for wound and pain management in livestock, it also has applications for human use. The commercial form was found to provide extended analgesia and promote wound healing. The current commercial form is a spray-on anaesthetic gel, applied by way of a spray applicator. The current commercial form can also be dripped onto a wound. Once sprayed or dripped onto a wound, the anaesthetic gel becomes very sticky and seals the wound. The current commercial form is a viscous liquid and is dispensed from a container at ambient temperature, without additional heating or cooling.

### DISCLOSURE OF INVENTION

The present inventors have now discovered that administering pain relieving compositions such as Tri-solfen™ (developed by Animal Ethics Pty Ltd) in the form of an aerosol spray can provide additional advantages. In particular, the inventors have discovered that administering pain relieving compositions such as Tri-solfen™ as a cooled aerosol spray can provide rapid onset local anaesthesia of intact skin (prior to procedures) as well as synergistic pain relief and haemostasis after wound creation due to an initial local refrigerant (cryoanaesthetic) effect and, there-after due to the pain relieving composition remaining on the wound.

The term “pain relieving aerosol spray” as used herein refers to an aerosol spray that can both prevent pain (eg. when applied to intact skin prior to a procedure) and relieve pain (eg. when applied to a wound or when remaining on the wound following a procedure). The term “pain relieving aerosol spray” is synonymous with “pain mitigating aerosol spray”.

According to a first aspect of the present invention, there is provided a pain relieving aerosol spray (pain relieving and pain preventing aerosol spray) comprising:

a pain relieving composition; and

at least one refrigerant.

According to a second aspect of the present invention, there is provided a pain relieving aerosol spray (pain relieving and pain preventing aerosol spray) comprising a pain relieving composition and at least one refrigerant, wherein the pain relieving aerosol spray is capable of providing rapid onset local anaesthesia of intact skin and/or a wound of a subject to which it is applied and further pain relief due to the pain relieving composition remaining on the wound of the subject.

According to a third aspect of the present invention, there is provided a pain relieving composition and at least one refrigerant that together are capable of producing a pain relieving aerosol spray (pain relieving and pain preventing aerosol spray).

According to a fourth aspect of the present invention, there is provided a pain relieving composition and at least one refrigerant that together are capable of producing a pain relieving aerosol spray (pain relieving and pain preventing aerosol spray), wherein the pain relieving aerosol spray is capable of providing rapid onset local anaesthesia of intact skin and/or a wound of a subject to which it is applied and further pain relief due to the pain relieving composition remaining on the wound of the subject.

According to a fifth aspect of the present invention, there is provided a method of cooling intact skin and/or a wound of a subject for pain relief and/or pain prevention, comprising the step of applying the pain relieving aerosol spray of any one of the first to fourth aspects of the invention onto intact skin and/or wound of the subject.

According to a sixth aspect of the present invention, there is provided a method of providing a subject with pain relief and/or pain prevention, said method comprising the step of applying the pain relieving aerosol spray of any one of the first to fourth aspects of the invention onto intact skin and/or wound of the subject.

According to a seventh aspect of the present invention, there is provided a method of providing a subject with rapid onset local anaesthesia and further pain relief, said method comprising the step of applying the pain relieving aerosol spray of any one of the first to fourth aspects of the invention onto intact skin and/or wound of the subject.

According to an eighth aspect of the present invention, there is provided a surgical procedure or animal husbandry procedure comprising the steps of:

1) applying the aerosol spray of any one of the first to fourth aspects of the invention to a subject to provide rapid onset local anaesthesia immediately prior to carrying out a surgical step or animal husbandry step on the subject that produces a wound; and/or

2) applying the aerosol spray of any one of the first to fourth aspects of the invention to a wound of the subject so as to provide rapid onset local anaesthesia and further pain relief.

According to a ninth aspect of the present invention, there is provided use of the pain relieving aerosol spray of any one of the first to fourth aspects of the invention in the preparation of a medicament for cooling skin and/or a wound of a subject for pain relief and/or pain prevention.

According to a tenth aspect of the present invention, there is provided use of the pain relieving aerosol spray of any one of the first to fourth aspects of the invention in the preparation of a medicament for providing a subject with pain relief and/or pain prevention.

According to an eleventh aspect of the present invention, there is provided use of the pain relieving aerosol spray of any one of the first to fourth aspects of the invention in the preparation of a medicament for providing a subject with rapid onset local anaesthesia and/or further pain relief.

According to a twelfth aspect of the present invention, there is provided use of at least one refrigerant and at least one pain relieving composition in the preparation of a medicament for providing rapid onset local anaesthesia and/or further pain relief to a subject.

According to a thirteenth aspect of the present invention, there is provided use of at least one refrigerant and at least one pain relieving composition in the preparation of a medicament for providing rapid onset local anaesthesia and/or further pain relief to a subject, wherein said medicament is formulated for administration to intact skin and/or a wound of the subject.

According to a fourteenth aspect of the present invention, there is provided a method of preparing a pain relieving aerosol spray comprising the step of combining at least one refrigerant with at least one pain relieving composition.

According to a fifteenth aspect of the present invention, there is provided a method of preparing a formulation capable of producing a pain relieving aerosol spray (pain relieving and pain preventing aerosol spray), said method comprising the step of combining at least one refrigerant with at least one pain relieving composition.

The term “wound” is to be understood as including: a minor cut, scratch, sting, burn and abrasion; and, a laceration, surgical incision, ulcer, (including inflammatory or infective skin lesions), penetrating wound, open fracture, major abrasion and major burn, including chemical burn.

The term “intact skin” is to be understood as not having: a minor cut, scratch, sting, burn or abrasion; or, a laceration, surgical incision, ulcer, (including inflammatory or infective skin lesions), penetrating wound, open fracture, major abrasion and major burn, including chemical burn.

Any suitable type of pain relieving composition can be used. The composition can be, for example, in the form of a liquid, ointment, gel, lotion, cream, crème, emulsion, paste, film or suspension—provided that it is sprayable.

Once applied, the pain relieving composition (or formulation) can be, for example, in the form of an adhesive/sticky/tenacious ointment, gel, lotion, crème, cream, emulsion, paste, solution or suspension, or may set and/or form a physical barrier, ‘skin’ or film.

Depending on the form of the composition (or formulation), the composition can include one or more of the following types of ingredients: adhesive; aqueous or oily diluent; carrier; excipient; base; buffer; pH adjuster; bittering agent (i.e. foul-tasting agent); suspending agent; thickening agent; gelling agent; viscosity increasing agent; emulsifier; emollient; humectant; stabilising agent; dispersing agent/dispersant; solubiliser; skin conditioning agent; skin protectant; skin penetration enhancer; fragrance; preservative; sunscreen agent; surfactant; textural modifier; colourant; and, waterproofing agent.

Any suitable ingredient amount can be used. In some embodiments, about 0.001 to 40 weight/volume % or weight/weight % or volume/volume % of ingredient is used (as well as all 0.001 increments between 0.001 and 40). In some embodiments, about 0.05 to 20% weight/weight or weight/volume of ingredient is used (as well as all 0.01 increments between 0.05 and 20). In some embodiments, about 0.1 to 10% weight/weight or weight/volume of ingredient is used (as well as all 0.01 increments between 0.1 and 10).

Suitable oily or aqueous bases, carriers, diluents and excipients are inert and physiologically acceptable and include, for example: bacteriostatic saline (saline containing benzyl alcohol), cetomacrogol, cetyl alcohol, glycerine, lanolin, petrolatum based creams, gels, hydrogels, saline, short chain alcohols and glycols (e.g. ethyl alcohol and propylene glycol), and water.

Suitable adhesives, thickening agents, gelling agents and/or viscosity increasing agents include: acrylamides copolymer, agarose, amylopectin, calcium alginate, calcium carboxymethyl cellulose, carbomer, carboxymethyl chitin, castor oil derivatives, cellulose gum, cellulosic preparation, cetyl alcohol, cetostearyl alcohol, dextrin, gelatin, hydroxy cellulose, hydroxyethylcellulose, hydroxypropylcellulose, hydroxpropyl starch, inert sugar, magnesium alginate, methylcellulose, microcrystalline cellulose, pectin, PEG's, polyacrylic acid, polymethacrylic acid, polyvinyl alcohol, quaternium ammonium compound of bentonite or zinc stearate, sorbitol, PPG's, sodium acrylates copolymer, sodium carrageenan, xanthum gum, and yeast beta-glucan.

Either water in oil or oil in water emulsions can be used. Examples of suitable surfactants and emulsifying agents include: non-ionic ethoxylated and non-ethoxylated surfactants, abietic acid, almond oil PEG, beeswax, butylglucoside caprate, C₁₈-C₃₆ acid glycol ester, C₉-C₁₅ alkyl phosphate, caprylic/capric triglyceride PEG-4 esters, cetomacrogol, ceteareth-7, cetereth-20, cetyl phosphate, cetyl stearyl alcohol, corn oil PEG esters, DEA-cetyl phosphate, dextrin laurate, dilaureth-7 citrate, dimyristyl phosphate, glycereth-17 cocoate, glyceryl erucate, glycerol, glyceryl laurate, G.M.S. acid stable, hydrogenated castor oil PEG esters, isosteareth-11 carboxylic acid, lecithin, lysolecithin, nonoxynol-9, octyldodeceth-20, palm glyceride, PEG diisostearate, PEG stearamine, poloxamines, polyglyceryls, potassium linoleate, PPGs, raffinose myristate, sodium caproyl lactylate, sodium caprylate, sodium cocoate, sodium isostearate, sodium tocopheryl phosphate, steareths, TEA-C₁₂-C₁₃ pareth-3 sulfate, tri-C₁₂-C₁₅ pareth-6 phosphate, and trideceths.

The composition can include one or more types of preservative. A suitable preservative, for example, can be: benzalkonium chloride, benzoic acid, benzothonium chloride, benzyl alcohol, 2-bromo-2-nitropropane-1,3-diol, bronopol, butylated hydroxyanisole, butylated hydroxytoluene, butyl paraben, chlorophene, chlorphenesin, diazolidinyl urea, DMDM hydantoin, ethyl paraben, formaldehyde-releasing preservative, hydroquinone, iodopropynyl butylcarbamate, imidazolidinyl urea, methyldibromo glutaronitrile, methylhydroquinone, methylisothiazolinone, methyl paraben, nitrosamines, o-cymen-5-ol, phenoxyethanol, propyl paraben, quaternium-15, sodium benzoate, sodium dehydroacetate, sodium hydroxymethylglycinate, sodium metabisulfite, and sodium sulfite.

Preferably, the composition includes the reducing agent sodium metabisulfite so as to enhance the stability of a vasoconstrictor (if present).

A skin conditioning agent, as defined herein, improves dry or damaged skin. Such agents, for example, include: acetyl cysteine, N-acetyl dihydrosphingosine, acrylates/behenyl acrylate/dimethicone acrylate copolymer, adenosine, adenosine cyclic phosphate, adensosine phosphate, adenosine triphosphate, alanine, albumen, algae extract, allantoin and deriviatives, aloe barbadensis extracts, aluminum PCA, amyloglucosidase, arbutin, arginine, azulene, bromelain, buttermilk powder, butylene glycol, caffeine, calcium gluconate, capsaicin, carbocysteine, carnosine, beta-carotene, casein, catalase, cephalins, ceramides, Chamomilla recutita (matricaria) flower extract, cholecalciferol, cholesteryl esters, coco-betaine, coenzyme A, corn starch modified, crystallins, cycloethoxymethicone, cysteine DNA, cytochrome C, darutoside, dextran sulfate, dimethicone copolyols, dimethylsilanol hyaluronate, DNA, elastin, elastin amino acids, epidermal growth factor, ergocalciferol, ergosterol, ethylhexyl PCA, fibronectin, folic acid, gelatin, gliadin, beta-glucan, glucose, glycine, glycogen, glycolipids, glycoproteins, glycosaminoglycans, glycosphingolipids, horseradish peroxidase, hydrogenated proteins, hydrolyzed proteins, jojoba oil, keratin, keratin amino acids, kinetin, lactoferrin, lanosterol, lauryl PCA, lecithin, linoleic acid, linolenic acid, lipase, lysine, lysozyme, malt extract, maltodextrin, melanin, methionine, mineral salts, niacin, niacinamide, oat amino acids, oryzanol, palmitoyl hydrolyzed proteins, pancreatin, papain, PEG, pepsin, phospholipids, phytosterols, placental enzymes, placental lipids, pyridoxal 5-phosphate, quercetin, resorcinol acetate, riboflavin, RNA, saccharomyces lysate extract, silk amino acids, sorbitol, sphingolipids, stearamidopropyl betaine, stearyl palmitate, tocopherol, tocopheryl acetate, tocopheryl linoleate, ubiquinone, Vitis vinifera (grape) seed oil, wheat amino acids, xanthan gum, and zinc gluconate.

The composition can include a skin penetration enhancer for enhancing the penetration of active ingredients, such as an anaesthetic agent. Any suitable type of enhancer can be used. Examples of suitable enhancers may include solvents, detergents or low carbon alcohols such as dimethylsulfoxide, oleyl alcohol, propylene glycol, methyl pyrrolidone and dodecylazyl cycloheptan 2-one.

The composition can comprise a hydrophilic or hydroalcoholic gelling agent. Any suitable amount of gelling agent can be used. Preferably, the composition comprises about 1 to 20 g per litre of at least one type of gum or cellulosic preparation (as well as all 0.1 g increments between 1 and 20). More preferably, the composition comprises a polyhydric alcohol in combination with a cellulosic preparation. Even more preferably, the composition comprises about 5 mg/mL hydroxy cellulose (eg. hydroxyethyl cellulose, ethylhydroxy cellulose) in combination with about 100 mg/mL non-crystallising liquid sorbitol (70%).

The composition (or formulation) is preferably capable of also promoting and prolonging contact of other active agents of the composition with the intact skin and/or wound.

The pain relieving composition can comprise any suitable type of pain relieving agent or agents. In some embodiments, an anaesthetic agent or combination of anaesthetic agents can be used. Examples of anaesthetic agents include: lignocaine, chloroprocaine, mepivacaine, bupivacaine, articaine, etidocaine, levobupivacaine, tetracaine, prilocaine, benzocaine, ropivacaine, cocaine, oxyprocaine, hexylcaine, dibucaine, piperocaine and procaine and pharmaceutically acceptable acids, bases and salts thereof.

Any suitable amount of pain relieving agent can be used. In some embodiments, about 0.001 to 20 weight/volume % or weight/weight % or volume/volume % of pain relieving agent is used (as well as all 0.001 increments between 0.001 and 20). In some embodiments, about 0.05 to 20% weight/weight or weight/volume of pain relieving agent is used (as well as all 0.01 increments between 0.05 and 20). In some embodiments, about 0.1 to 10% weight/weight or weight/volume of pain relieving agent is used (as well as all 0.01 increments between 0.1 and 10).

Examples of other potentially suitable anaesthetic agents include: butamben, butambenpicrate, dimethisoquin hydrochloride, diperodon, diphenhydramine, dyclonine, ketamine, methapyriline, p-buthylaminobenzoic acid, 2-(die-ethylamino) ethyl ester hydrochloride, pramoxine and tripelennamine.

The composition preferably provides maximum anaesthesia with minimal risk of toxicity. The formulation or the composition can be varied, as required, for potency, speed of onset and duration of anaesthetic action.

In some embodiments, the composition comprises at least one local anaesthetic agent. In some embodiments, the composition comprises at least one local anaesthetic agent having a rapid onset of action. In some embodiments, the composition comprises at least one local anaesthetic agent having a long duration of action. In some embodiments, the composition comprises both at least one local anaesthetic agent having a rapid onset of action and at least one local anaesthetic agent having a long duration of action. It is to be understood that some local anaesthetic agents can provide both a rapid onset of action and long duration of action, such as tetracaine/amethocaine, so the local anaesthetic agent providing a rapid onset of action and local anaesthetic agent providing a long duration of action can be one and the same.

Anaesthetic agents that usually have a rapid onset of action (usually between about 20 seconds to 5 minutes) include lignocaine, prilocaine, amethocaine and cocaine.

Anaesthetic agents that have a much greater duration of action (usually between about 4-12 hours of anaesthesia) include bupivacaine and amethocaine/tetracaine. Bupivacaine may typically provide up to about 6-12 hours of anaesthesia, depending on the method of administration.

Any suitable amount of anaesthetic agent can be used in the composition but preferably about 0.01-10 weight/volume % of anaesthetic agent is used (as well as all 0.01 increments between 0.01 and 10, eg. 0.01, 0.02 etc).

Any suitable amount of rapid onset anaesthetic agent can be used in the composition but preferably about 0.01-10 weight/volume % of anaesthetic agent is used (as well as all 0.01 increments between 0.01 and 10). Preferably, about 2-8 weight/volume % anaesthetic agent is used in those situations where a rapid onset of action is required (as well as all 0.01 increments between 2 and 8). More preferably, about 5% weight/volume anaesthetic agent is used.

In some embodiments, about 1-10 weight/volume % lignocaine is used (as well as all 0.01 increments between 1 and 10, eg. 0.01, 0.02 etc). In some embodiments, about 2-8 weight/volume % lignocaine is used as the anaesthetic agent in those situations where a rapid onset of action is required (as well as all 0.01 increments between 2 and 8). In some embodiments, about 5% lignocaine is used.

Any suitable amount of long duration of action anaesthetic agent can be used in the composition but preferably about 0.01-10 weight/volume % of anaesthetic agent is used (as well as all 0.01 increments between 0.01 and 10). Preferably, about 0.1-5 weight/volume % anaesthetic agent is used in those situations where a long duration of action is required (as well as all 0.01 increments between 0.1 and 5). More preferably, about 0.5% weight/volume anaesthetic agent is used.

In some embodiments, the composition can comprise any suitable amount of bupivacaine if lignocaine has an inadequate duration of action. Preferably, the composition comprises about 0.1-5 weight/volume % bupivacaine (as well as all 0.01 increments between 0.1 and 5), and more preferably about 0.5% bupivacaine.

In some embodiments, about 0.5-10 weight/volume % tetracaine is used (as well as all 0.01 increments between 0.5 and 10, eg. 0.51, 0.52 etc). In some embodiments, about 1-5 weight/volume % tetracaine is used as the anaesthetic agent, usually the sole anaesthetic agent.

In some embodiments, at least one analgesic agent and/or at least one anti-inflammatory agent can be used. Examples of potentially suitable analgesic and/or anti-inflammatory agents include: meloxicam, acetaminophen, aspirin, salicylic acid, methyl salicylate, choline salicylate, glycol salicylate, 1-menthol, camphor, mefenamic acid, fluphenamic acid, indomethacin, diclofenac, alclofenac, ibuprofen, carprofen, ketoprofen, naproxene, pranoprofen, fenoprofen, sulindac, fenbufen, clidanac, flurbiprofen, indoprofen, protizidic acid, fentiazac, tolmetin, tiaprofenic acid, bendazac, bufexemacpiroxicam, phenylbutazone, oxyphenbutazone, clofezone, pentazocine, mepirizole, hydrocortisone, cortisone, dexamethasone, fluocinolone, triamcinolone, medrysone, prednisolone, flurandrenolide, prednisone, halcinonide, methylprednisolone, fludrocortisone, corticosterone, paramethasone, betamethasone, ibuprophen, naproxen, fenoprofen, fenbufen, flurbiprofen, indoprofen, ketoprofen, suprofen, indomethacin, piroxicam, aspirin, salicylic acid, diflunisal, methyl salicylate, phenylbutazone, sulindac, mefenamic acid, meclofenamate sodium, and tolmetin.

Any suitable amount of analgesic or anti-inflammatory agent can be used in the composition but preferably about 0.01-10 weight/volume % of agent is used (as well as all 0.01 increments between 0.01 and 10).

The composition can further include a vasoconstrictor to decrease the rate of vascular absorption of the pain relieving agent, so to improve the depth and duration of pain relieving agent, to reduce bleeding from a wound of the subject, as well as to reduce systemic toxicity. Any suitable type of vasoconstrictor can be used. Suitable vasoconstrictors include, for instance, adrenaline (epinephrine), noradrenalin (norepinephrine) and fenylpressin. Preferably, the composition includes about 1:1000-1:10,000 vasoconstrictor (as well as all 10 factor increments between 1000 to 10,000), and more preferably 1:2,000 vasoconstrictor. Preferably the vasoconstrictor is adrenaline.

The composition can include one or more other active ingredients. An active ingredient, as defined herein, is a compound that provides benefit to the subject. The active ingredient can be, for instance, an antibody, anticoagulant, antiproliferative, cytokine, cytotoxin, growth factor, interferon, haemostatic agent, hormone, lipid, demineralized bone or bone morphogenetic protein, cartilage inducing factor, oligonucleotide, polymer, polysaccharide, polypeptide, protease inhibitor, vitamin, mineral, antiseptic agent, insecticide or insect repellent, antibiotic, antiparasitic or antifungal agent.

Any suitable amount of active ingredient can be used. In some embodiments, about 0.001 to 20 weight/volume % or weight/weight % or volume/volume % of active ingredient is used (as well as all 0.001 increments between 0.001 and 20). In some embodiments, about 0.05 to 20% weight/weight or weight/volume of active ingredient is used (as well as all 0.01 increments between 0.05 and 20). In some embodiments, about 0.1 to 10% weight/weight or weight/volume of active ingredient is used (as well as all 0.01 increments between 0.1 and 10).

The composition preferably includes an antiseptic agent to, amongst other things, minimize skin and/or wound contamination and infection. Any suitable type of antiseptic agent can be used. Examples of suitable antiseptic agents include quaternary ammonium salts. Suitable antiseptic agents include cetrimide, povidone-iodine, chlorhexidine, iodine, benzalkonium chloride, benzoic acid, nitrofurazone, benzoyl peroxide, hydrogen peroxide, hexachlorophene, phenol, resorcinol and cetylpyridinium chloride. A preferred example is cetrimide, which is a mixture of different quaternary ammonium salts including cetrimonium bromide (CTAB).

Any suitable amount of antiseptic agent can be used. Preferably the composition comprises anywhere between approximately 0.01 weight/weight (or weight/volume or volume/volume) % and approximately 15 weight/weight (or weight/volume or volume/volume) % of antiseptic agent, which includes all 0.01 increments between 0.01 and 15%, including 0.02, 0.03 etc.

The composition can include an insecticide or insect repellent to stop insects from infesting a wound of the subject. Any suitable type of insecticide or insect repellent can be used. Examples of suitable insecticides include: trichlorfon, triflumeron, fenthion, bendiocarb, cyromazine, dislubenzuron, dicyclanil, fluazuron, amitraz, deltamethrin, cypermethrin, chlorfenbinphos, flumethrin, ivermectin, abermectin, avermectin, doramectin, moxidectin, zeti-cypermethrin, diazinon, spinosad, imidacloprid, nitenpyran, pyriproxysen, sipronil, cythioate, lufenuron, selamectin, milbemycin oxime, chlorpyrifos, coumaphos, propetamphos, alpha-cypermethrin, high cis cypermethrin, ivermectin, diflubenzuron, cyclodiene, carbamate and benzoyl urea.

The composition can comprise a colouring agent/colourant/marker such that application of the composition to the skin and/or wound can be easily assessed by eye. The colourant can be a pigment and/or dye. Suitable colourants include, for example, common food dyes or the ORCODERM®, ORCOBRITE® and ORCOFUR® lines of pigments and dyes sold by the Organic Dyestuffs Corporation. Preferably, the colourant is non-toxic and will not permanently stain the skin or animal hide.

In a first preferred embodiment, the pain relieving composition comprises:
about 100 mg/mL non-crystallising liquid sorbitol (70%);
about 50.0 mg/mL lignocaine HCl;
about 5.0 mg/mL bupivacaine HCl;
about 1.5 mg/mL sodium metabisulfite;
about 5.0 mg/mL cetrimide;
about 45.0 μg/mL adrenaline tartrate;
about 5.0 mg/mL hydroxy cellulose; and optionally
dye.

In a second preferred embodiment, the pain relieving composition comprises:
about 100 mg/mL non-crystallising liquid sorbitol (70%);
about 40.0 mg/mL lignocaine HCl;
about 1.5 mg/mL sodium metabisulfite;
about 5.0 mg/mL cetrimide;
about 36.0 μg/mL adrenaline tartrate;
about 5.0 mg/mL hydroxy cellulose; and optionally
dye.

In a third preferred embodiment, the pain relieving composition comprises:

| Ingredient | % w/v | g/500 mL |
|---|---|---|
| Water | ~87 | ~435 |
| Cetrimide | ~0.5 | ~2.5 |
| Hydroxyethylcellulose | ~0.5 | ~2.5 |
| Tetracaine HCl | ~5 | ~25.2 |
| Sodium metabisulfite | ~0.15 | ~0.75 |
| Adrenaline acid tartrate | ~0.00495 | ~0.025 |
| Sorbitol 70% | ~10 | ~50 |
| Dye | ~0.005 | ~0.025 |

Final pH 4.0

In a fourth preferred embodiment, the pain relieving composition comprises:

| Ingredient | % w/v | g/500 mL |
|---|---|---|
| Water | ~90 | ~450 |
| Cetrimide | ~0.50 | ~2.5 |
| Hydroxyethylcellulose | ~0.5 | ~2.5 |
| Tetracaine HCl | ~1 | ~10.1 |
| Sodium metabisulfite | ~0.15 | ~0.75 |
| Adrenaline acid tartrate | ~0.00495 | ~0.025 |
| Sorbitol 70% | ~10 | ~50 |
| Dye | ~0.005 | ~0.025 |

Citric acid can be used to adjust the pH to about 4.0.

‘Refrigerant’ as used herein is a volatile liquid that evaporates on contact with the skin or wound and/or a pressurised gas that when contacting the skin or wound causes a local refrigerant effect whereby the skin or wound is cooled, chilled or frozen. In this way, the refrigerant can provide local anaesthesia, such as for burns, incisions and other wound types caused by surgical and animal husbandry procedures. Rapid evaporation of the volatile liquid from the skin or wound or cold gas striking the skin or wound's surface causes a drop in temperature and results in temporary interruption of pain sensation.

The pain relieving aerosol spray can comprise any suitable type of refrigerant. The pain relieving aerosol spray can comprise one type of refrigerant or more than one type of refrigerant. The refrigerant can be a gas. The refrigerant can be a volatile liquid. The at least one refrigerant can be flammable or non-flammable. The pain relieving aerosol spray can comprise 1, 2, 3, 4, 5 or even more types of refrigerants. In some embodiments, the pain relieving aerosol spray can comprise a blend or mixture of two or more refrigerants. In some embodiments, the 2 or more refrigerants can either be a combination of gas and gas, volatile liquid and gas, or volatile liquid and volatile liquid.

Examples of suitable refrigerants include any one or more of the following:

a compressed gas such as an inert gas, such as nitrogen, carbon dioxide, nitrous oxide, oxygen or air;

a liquefied hydrocarbon such as methane, ethane, ethyl alcohol, propane, butane, n-butane, isobutane, pentane, isopentane, n-pentane; a mixture of 2, 3, 4 or more hydrocarbons (eg. a mixture of n-butane, isobutane and propane, or a mixture of propane and butane);

a fluorinated hydrocarbon such as trichloromonofluromethane, dichlorodifluoromethane, dichlorotetrafluroethane, 1,1,1,3,3 pentafluoropropane or 1,1,1,2 Tetrafluoroethane; liquid nitrogen;

an ether such as dimethyl ether (DME) or methyl ethyl ether;

a hydrofluoroalkane (HFA) such as HFA 134a (1,1,1,2,-tetrafluoroethane) or HFA 227 (1,1,1,2,3,3,3-heptafluoropropane); or

a combination of these.

The pain relieving aerosol spray can comprise any suitable amount of refrigerant. Preferably the pain relieving aerosol spray comprises anywhere between approximately 10 and approximately 99.9 weight/weight (or weight/volume or volume/volume) % of refrigerant, which includes all 0.1 increments between 10 and 99.5%, including 10.5, 11, 11.5.

In some embodiments the pain relieving aerosol spray comprises between approximately 20% weight/weight and 80% weight/weight refrigerant. In some embodiments the pain relieving aerosol spray comprises between approximately 30% weight/weight and approximately 70% weight/weight refrigerant. In some embodiments, the pain relieving aerosol spray comprises approximately 30%, 35%, 40%, 45%, 50%, 55%, 60%, 65%, 70%, 75% or 80% weight/weight refrigerant. More preferably, the pain relieving aerosol spray comprises approximately 30%, 35%, 40%, 45%, 50%, 55%, 60%, 65%, 70%, 75% or 80% weight/weight hydrocarbon/s or ether/s.

The aerosol spray can be in the form of a sprayable stream, sprayable mist or sprayable foam. The aerosol spray can comprise or can be delivered from a pressurised spray container or can, in which case it may contain at least one propellant. In some embodiments the at least one refrigerant can function as the at least one propellant. The aerosol spray can further comprise at least one solvent for the propellant, but this will depend on the nature of the propellant.

In some embodiments the aerosol spray comprises a gaseous suspension of liquid particles. In some embodiments the aerosol spray comprises an aerosol mist comprising liquid particles. In some embodiments the aerosol spray comprises a foam, whereby the foam comprises gas pockets trapped in liquid. Most preferably, the aerosol spray is in the form of a sprayable foam.

In some embodiments the pain relieving composition is in the form of a liquid that is expelled from the pressurised container as a foam and sets as a sticky viscous gel when exposed to the skin or wound, after the refrigerant evaporates or otherwise dissipates.

The aerosol spray can comprise a delivery nozzle, cap, tip or actuator such as a standard nozzle or cap as shown in FIG. 1 or a needle cap or needle nozzle as shown in FIG. 1.

The inventors have found that both the formulation and cap/nozzle type can have a bearing on the foam properties of the aerosol spray when delivered to the skin or wound.

Any suitable type of propellant or blend of propellants can be used. The propellant or propellant blend can be flammable or non-flammable. The propellant can be a compressed gas, soluble gas or liquefied gas. The propellant can also act as solvent, diluent, viscosity modifier or freezant.

Examples of suitable propellants include any one or more of the following:

a compressed gas such as an inert gas, such as nitrogen, carbon dioxide, nitrous oxide, oxygen or air;

a liquefied hydrocarbon such as methane, ethane, ethyl alcohol, propane, butane, n-butane, isobutane, pentane, isopentane, n-pentane; a mixture of 2, 3, 4 or more hydrocarbons (eg. a mixture of n-butane, isobutane and propane, or a mixture of propane and butane);

a fluorinated hydrocarbon such as trichloromonofluromethane, dichlorodifluoromethane, dichlorotetrafluroethane, 1,1,1,3,3 pentafluoropropane or 1,1,1,2 Tetrafluoroethane;

liquid nitrogen;

an ether such as dimethyl ether (DME) or methyl ethyl ether; or

a hydrofluoroalkane (HFA) such as HFA 134a (1,1,1,2,-tetrafluoroethane) or HFA 227 (1,1,1,2,3,3,3-heptafluoropropane); or

a combination of these.

The aerosol spray can comprise any suitable amount of propellant. Preferably the aerosol spray comprises anywhere between approximately 10 and approximately 99.9 weight/weight (or weight/volume or volume/volume) % of propellant, which includes all 0.1 increments between 10 and 99.5%, including 10.5, 11, 11.5 etc.

In some preferred embodiments, the refrigerant is carbon dioxide or other type of compressed gas, which also functions as the propellant. In some preferred embodiments refrigerant and/or propellant is used to balance in the aerosol container, particularly when a compressed gas.

The pain relieving aerosol spray can be applied for any suitable period of time. The time period will typically be between about 1 and 10 seconds, although it may be shorter or longer (eg. up to 15, 20, 25 or 30 seconds). Preferable application times include, but are not limited to, 1, 1.5, 2, 2.5, 3, 3.5, 4, 4.5, 5, 5.5, 6, 6.5, 7, 7.5, 8, 8.5, 9, 9.5 and 10 seconds.

The pain relieving aerosol spray can either cool, chill or freeze the skin or wound, but preferably cools it to a temperature from between about −20° C. up to about 10° C., including −20, −19.5, −19 . . . etc. . . . 0, 0.5, 1, 1.5, 2, 2.5, 3 . . . etc. . . . 7, 7.5, 8, 8.5, 9, 9.5 and 10° C. In some embodiments the pain relieving aerosol spray can cool the skin or wound to a temperature of between about 1 and 2° C. In some embodiments the pain relieving aerosol spray can cool the skin or wound to a temperature below about 9° C. or 10° C.

The subject can be a human. The subject can be another type of mammal or animal. The subject can be a farm animal or livestock, such as a sheep, horse, cow, goat or pig. The subject can be a companion animal, such as a cat or dog. The subject can be a laboratory animal, such as a mouse, rat or rabbit. The subject can be an animal such as a sheep/lamb, horse, cow/calf, goat, pig/piglet, dog or cat. The subject can be another type of animal.

The aerosol spray can be used for an animal husbandry procedure. The procedure can be, for example, castration, mulesing, shearing, ear tagging, branding, hot branding, dehorning, hot dehorning, dis-budding, marking, or treating a wound such as an open wound, eg. caused by accident or surgery (laceration, incision, ulcer or burn).

Other properties and advantages of the pain relieving composition (particularly Tri-solfen™) are described in U.S. Pat. Nos. 8,822,416, 8,960,128 and 9,592,318, the entire contents of which are incorporated herein by way of reference.

Preferably, the aerosol spray or pain relieving composition is in the form of a liquid prior to having been applied skin or a wound. Preferably, the aerosol spray or pain relieving composition forms, or is in the form of, a sticky, viscous, adhesive gel when applied to skin or a wound. Preferably, the aerosol spray or pain relieving composition is in the form of a spray-on gel that can coat skin or the wound of the subject and can maximise delivery of active ingredients to the skin or wound by way of staying moist and viscous (i.e. “sticky”).

Preferably, the aerosol spray or pain relieving composition forms an effective long-lasting barrier over the skin or wound. The term “long-lasting barrier” is to be understood as meaning a barrier/seal that is substantially capable of remaining intact over a wound for hours, days, a week or even weeks, or until the wound has naturally sealed or the pain has otherwise abated by way of the natural healing process—eg. about 1, 2, 3, 4, 5, 6, 7, 8, 9, 10, 11, 12, 13, 14, 15, 16, 17, 18, 19, 20, 21, 22, 23 or 24 hours, or about one, two, three, four, five, six or seven days, or one, two, three, four or more weeks.

The barrier preferably aids in the healing process, presumably by minimising or preventing water loss from the wound and by acting as a barrier against microbial contamination.

Preferably the aerosol spray or pain relieving composition is in the form of a liquid that thickens to an adhesive gel when reacting with physiological fluids of the wound.

Preferably the aerosol spray or pain relieving composition is capable of coating and adhering to the wound.

Preferably the aerosol spray or pain relieving composition is biocompatible and absorbable such that they do not require removal.

In some embodiments, the aerosol spray or pain relieving composition is applied as a liquid to a wound, such as a spray-on liquid.

In some embodiments, the aerosol spray or pain relieving composition forms an adhesive gel when applied to a wound.

In some embodiments, the aerosol spray or pain relieving composition forms a long-lasting barrier over a wound.

In some embodiments, the aerosol spray or pain relieving composition comprises an antiseptic, such as cetrimide.

In some embodiments, the aerosol spray or pain relieving composition comprises a reducing agent or preservative, such as sodium metabisulfite.

In some embodiments, the aerosol spray or pain relieving composition comprises a gelling agent or thickener, such as hydroxyethyl cellulose.

In some embodiments, the aerosol spray or pain relieving composition comprises a gelling agent or thickener, such as non-crystallising liquid sorbitol (70%).

In some embodiments, the aerosol spray or pain relieving composition comprises a colourant, such as a dye.

In some embodiments, the aerosol spray or pain relieving composition comprises a pH adjuster or buffering agent.

In some embodiments, the pain relieving composition comprises: a liquid gel matrix that contains the following: lidocaine; adrenalin; and cetrimide, and the composition is optionally coloured; or a liquid gel matrix that contains the following: tetracaine; adrenalin; and cetrimide, and the composition is optionally coloured; or a liquid gel matrix that contains the following: lidocaine; bupivacaine; adrenalin; and cetrimide, and the composition is optionally coloured, wherein said composition has a pH lower than about 4.0.

In a first preferred embodiment, the pain relieving composition (“Composition 1”) comprises:

about 100 mg/ml non-crystallising liquid sorbitol (70%);

about 50.0 mg/ml lignocaine HCl;

about 5.0 mg/ml bupivacaine HCl;

about 1.5 mg/ml sodium metabisulfite;

about 5.0 mg/ml cetrimide;

about 45.0 μg/ml adrenaline tartrate;

about 5.0 mg/ml hydroxy cellulose; and optionally

colourant such as a dye.

If desired, lignocaine can be swapped out and tetracaine swapped in at about 1-10%, but preferably about 5% (50 mg/ml) (“Composition 2”).

In a second preferred embodiment, the pain relieving composition (“Composition 3”) comprises:

about 100 mg/ml non-crystallising liquid sorbitol (70%);

about 40.0 mg/ml lignocaine HCl;

about 1.5 mg/ml sodium metabisulfite;

about 5.0 mg/ml cetrimide;

about 36.0 μg/ml adrenaline tartrate;

about 5.0 mg/ml hydroxy cellulose; and optionally

colourant such as a dye.

If desired, lignocaine can be swapped out and tetracaine swapped in at about 1-10%, but preferably about 5% (50 mg/ml) (“Composition 4”).

In a third preferred embodiment, the pain relieving composition (“Composition 5”) comprises:

about 100.0 mg/ml purified water sorbitol liquid 70% non-crystallising;

about 50.0 mg/ml (5%) tetracaine HCl;

about 1.5 mg/ml sodium metabisulfite;

about 5.0 mg/ml cetrimide;

about 45.0 μg/ml adrenaline tartrate;

about 5.0 mg/ml hydroxy cellulose; and optionally

colourant such as a food dye (quantity to suit (q.s.)).

In a fourth preferred embodiment, the pain relieving composition (“Composition 6”) comprises: lignocaine, bupivacaine, adrenaline, cetrimide, 2-ethyl hydroxycellulose, sodium metabisulfite, liquid sorbitol (70%), buffer, and, optionally colourant such as a dye.

If desired, lignocaine can be swapped out and tetracaine swapped in at about 1-10%, but preferably about 5% (50 mg/ml) (“Composition 7”).

In a fifth preferred embodiment, the pain relieving composition (“Composition 8”) comprises: amethocaine/tetracaine, adrenaline, cetrimide, 2-ethyl hydroxycellulose, sodium metabisulfite, liquid sorbitol (70%), buffer, and, optionally, colourant such as a dye.

In a preferred embodiment, the composition (“Composition 9”) is as described or substantially as described in U.S. Pat. Nos. 8,960,128, 8,822,416 and 9,592,318 (to Animal Ethics Pty Ltd), the entire contents of which are incorporated herein by way of reference.

Having broadly described the invention in its various embodiments, non-limiting examples of embodiments will now be given.

### DESCRIPTION OF THE FIGURES

FIG. 1 shows various nozzles and caps for an aerosol can, for delivering the aerosol spray.

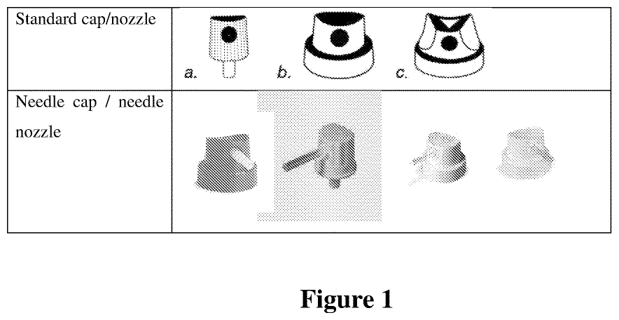

### DESCRIPTION OF PREFERRED EMBODIMENTS

### Example 1—Formulation of a Pain Relieving Composition Having a Long Duration of Action

This Example describes the preparation of a particularly preferred topical analgesic pain relieving composition. The composition is in the form of a gel that provides a prolonged analgesic effect. The composition has the following formulation:

| | | |
|---|---|---|
| Sorbitol Liquid 70% Non-Crystallising | 100.0 | mg/mL |
| Lignocaine HCl | 50.0 | mg/mL (5%) |
| Bupivacaine HCl | 5.0 | mg/mL (0.5%) |
| Sodium Metabisulfite | 1.5 | mg/mL |
| Cetrimide | 5.0 | mg/mL |
| Adrenaline Tartrate | 45.0 | μg/mL |
| Hydroxy Cellulose | 5.0 | mg/mL |

| | |
|---|---|
| Purified water | to 1 mL |
| Optional: | |
| Food Dye (e.g. brilliant blue) | Quantity to suit (q.s.) |

The composition is prepared by combining/blending the above ingredients to achieve the required consistency.

If desired, the composition can further comprise an anti-inflammatory agent (e.g. meloxicam or carprofen), and/or an insecticide/insect repellent such as diazinon, spinosad or cyromazine (at about 1 mg/mL), and/or a skin penetrating enhancer, and/or a bittering agent.

### Example 2—Formulation of a Pain Relieving Composition

This Example describes the preparation of another preferred pain relieving composition. The composition is in most respects the same as the composition of Example 1, except that it excludes bupivacaine. The composition has the following formulation:

| | | |
|---|---|---|
| Sorbitol Liquid 70% Non-Crystallising | 100.0 | mg/mL |
| Lignocaine HCl | 40.0 | mg/mL (4%) |
| Sodium Metabisulfite | 1.5 | mg/mL |
| Cetrimide | 5.0 | mg/mL |
| Adrenaline Tartrate | 36.0 | μg/mL (1:2000) |

| | |
|---|---|
| Hydroxy Cellulose | q.s. |
| Purified water | to 1 mL |
| Optionally: | |
| Food Dye (e.g. blue) | q.s. |

The composition is prepared by combining/blending the above ingredients to achieve the required consistency.

If desired, the composition can further comprise an anti-inflammatory agent (e.g. meloxicam or carprofen), and/or an insecticide/insect repellent such as diazinon, spinosad or cyromazine (at about 1 mg/mL), and/or a skin penetrating enhancer, and/or a bittering agent.

### Example 3—Formulation of a Pain Relieving Composition

This Example describes the preparation of another preferred pain relieving composition having only tetracaine as the anaesthetic agent. The composition has the following formulation:

| Ingredient | % w/v | g/500 mL |
|---|---|---|
| Water | ~87 | ~435 |
| Cetrimide | ~0.5 | ~2.5 |
| Hydroxyethylcellulose | ~0.5 | ~2.5 |
| Tetracaine HCl | ~5 | ~25.2 |
| Sodium metabisulfite | ~0.15 | ~0.75 |
| Adrenaline acid tartrate | ~0.00495 | ~0.025 |
| Sorbitol 70% | ~10 | ~50 |
| Dye | ~0.005 | ~0.025 |

Final pH 4.0

The composition is prepared by combining/blending the above ingredients to achieve the required consistency.

If desired, the composition can further comprise an anti-inflammatory agent (e.g. meloxicam or carprofen), and/or an insecticide/insect repellent such as diazinon, spinosad or cyromazine (at about 1 mg/mL), and/or a skin penetrating enhancer, and/or a bittering agent.

### Example 4—Formulation of a Pain Relieving Composition

This Example describes the preparation of another preferred pain relieving composition. The composition is in most respects the same as the composition of Example 3. The composition has the following formulation:

| Ingredient | % w/v | g/500 mL |
|---|---|---|
| Water | ~90 | ~450 |
| Cetrimide | ~0.50 | ~2.5 |
| Hydroxyethylcellulose | ~0.5 | ~2.5 |
| Tetracaine HCl | ~1 | ~10.1 |
| Sodium metabisulfite | ~0.15 | ~0.75 |
| Adrenaline acid tartrate | ~0.00495 | ~0.025 |
| Sorbitol 70% | ~10 | ~50 |
| Dye | ~0.005 | ~0.025 |

Required a small amount of citric acid to adjust the pH to about 4.0.

The composition is prepared by combining/blending the above ingredients to achieve the required consistency.

If desired, the composition can further comprise an anti-inflammatory agent (e.g. meloxicam or carprofen), and/or an insecticide/insect repellent such as diazinon, spinosad or cyromazine (at about 1 mg/mL), and/or a skin penetrating enhancer, and/or a bittering agent.

### Example 5—Formulation of a Topical Anaesthetic Crème Having a Long Duration of Action

This Example describes the preparation of another topical pain relieving composition in the form of a crème. The composition has the following formulation:

| | |
|---|---|
| Cetyl Alcohol | 78.00 mg/mL |
| Paraffin Wax | 135.00 mg/mL |
| Glycerol | 75.00 mg/mL |
| Lauryl Sulfate | 10.00 mg/ml |
| Lignocaine HCl | 50.00 mg/mL |
| Bupivacaine HCl | 5.00 mg/mL |
| Sodium Metabisulfite | 1.50 mg/mL |
| Cetrimide | 5.00 mg/mL |
| Hydrochloric Acid 25% | q.s. |
| Adrenaline Acid Tartare | 0.045 mg/mL |
| Purified Water | to 1 mL |

The composition is prepared by combining the above ingredients to achieve the required consistency as required. The composition is in the form of a “sticky” crème.

If desired, the composition can further comprise an anti-inflammatory agent, and/or an insecticide/insect repellent, and/or a skin penetrating enhancer, and/or a bittering agent.

### Example 6—Formulation of a Topical Anaesthetic Gel Having a Long Duration of Action

This example describes the preparation of another topical anaesthetic composition having a gum base. The composition has the following formulation:

| | |
|---|---|
| Xanthum Gum | 10.00 mg/mL |
| Gum Arabic | 1.00 mg/mL |
| Sorbitol Liquid | 100.00 mg/mL |
| Lignocaine HCl | 50.00 mg/mL |
| Bupivacaine HCl | 5.00 mg/mL |
| Sodium Metabisulfite | 1.50 mg/mL |
| Cetrimide | 5.00 mg/mL |
| Hydrochloric Acid 25% | q.s. |
| Adrenaline Acid Tartare | 0.045 mg/mL |
| Purified Water | to 1 mL |
| Optional: | |
| Dye | q.s. |

The composition is prepared by combining the above ingredients to achieve the required consistency. The composition is in the form of a “sticky” gel.

If desired, the composition can further comprise an anti-inflammatory agent, and/or an insecticide/insect repellent, and/or a skin penetrating enhancer, and/or a bittering agent.

### Example 7—Formulation of a Topical Anaesthetic Gel Having a Long Duration of Action

This Example describes the preparation of another topical anaesthetic composition having a polyacrylic acid base. The composition has the following formulation:

| | |
|---|---|
| Polyacrylic Acid | 10.00 mg/mL |
| Sodium Hydroxide | q.s. |
| Polyhydrogenated Castor Oil | 10.00 mg/mL |
| Sorbitol Liquid | 100.00 mg/mL |
| Lignocaine HCl | 50.00 mg/mL |
| Bupivacaine HCl | 5.00 mg/mL |
| Sodium Metabisulfite | 1.50 mg/mL |
| Cetrimide | 5.00 mg/mL |
| Hydrochloric Acid 25% | q.s. |
| Adrenaline Acid Tartare | 0.045 mg/mL |
| Purified Water | to 1 mL |
| Optional: | |
| Dye | q.s. |

The composition is prepared by combining the above ingredients to achieve the required consistency. The composition is in the form of a “sticky” gel.

If desired, the composition can further comprise an anti-inflammatory agent, and/or an insecticide/insect repellent, and/or a skin penetrating enhancer, and/or a bittering agent.

### Example 8—Formulation of a Topical Anaesthetic Gel Having an Insecticide and a Skin Penetrating Enhancer

This Example describes the preparation of another topical anaesthetic composition in the form of a spray-on gel having an insecticide (spinosad) as well as a skin penetrating enhancer (propylene glycol). The composition has the following formulation:

| | |
|---|---|
| Cellulose | 5.00 mg/mL |
| Spinosad | 1.25 mg/mL |
| Propylene Glycol | 100.00 mg/mL |
| Sorbitol Liquid | 50.00 mg/mL |
| Lignocaine HCl | 50.00 mg/mL |
| Bupivacaine HCl | 5.00 mg/mL |
| Sodium Metabisulfite | 1.50 mg/mL |
| Cetrimide | 5.00 mg/mL |
| Hydrochloric Acid 25% | q.s. |
| Adrenaline Acid Tartare | 0.045 mg/mL |
| Purified Water | to 1 mL |
| Optional: | |
| Dye | q.s. |

The composition is prepared by combining the above ingredients to achieve the required consistency. The composition is in the form of a “sticky” gel”.

If desired, the composition can further comprise an anti-inflammatory agent, and/or a bittering agent.

### Example 9—Preparation of Aerosol Sprays Utilising Carbon Dioxide as the Refrigerant and Propellant

This Example describes the preparation of aerosol sprays comprising the pain relieving composition of any one of Examples 1 to 8 in combination with carbon dioxide as the refrigerant/propellant.

An aerosol container is partially filled with the pain relieving composition of any one of Examples 1 to 8. The container is then sealed and charged with carbon dioxide until suitably pressurised. The carbon dioxide serves both as a refrigerant and propellant. The container's composition comprises 20-50% pain relieving composition (weight/weight) and carbon dioxide to balance.

Pressing an actuator button opens a valve of the container such that pressurised carbon dioxide can force the pain relieving composition up a dip tube of the container and through the valve and cap/nozzle. The pain relieving composition can be applied as an aerosol mist or foam depending on the final composition. A specially articulated spray nozzle (eg. needle cap) can also be used, if required, to help produce a long-lasting foam.

### Example 10—Preparation of Aerosol Sprays Utilising Liquified Hydrocarbon or Dimethyl Ether as the Refrigerant and Propellant

An aerosol container is partially filled with the pain relieving composition of any one of Examples 1 to 8. The container is then sealed and charged with hydrocarbon until suitably pressurised. The hydrocarbon serves both as a refrigerant and propellant. The container's composition comprises 20 to 50% pain relieving composition (weight/weight) and 50-80% w/w butane and propane blend, butane, propane and isobutane blend, or dimethyl ether.

Preferred formulations and properties are shown in Table 1 below.

| | | Pain | | |
|---|---|---|---|---|
| | | Relieving | | |
| Formulation | Refrigerant/Propellant | Composition | Cap/Nozzle | |
| No. | (% w/w) | (% w/w) | Type | Comments/Properties |
| 1 | Butane and propane blend | Examples 1 to 4 | Needle cap | Delivered as sticky |
| | 50 | 20-50 | | long-lasting foam |
| | | | | with substantially no |
| | | | | run-off from the wound. |
| 2 | Butane, propane and | Examples 1 to 4 | Needle cap | Delivered as sticky |
| | isobutane blend | 20-50 | | long-lasting foam |
| | 50 | | | with substantially no |
| | | | | run-off from the wound |
| 3 | Dimethyl ether | Examples 1 to 4 | Needle cap | Delivered as sticky |
| | 50 | 20-50 | | short-lived foam with |
| | | | | substantially no run- |
| | | | | off from the wound |
| 4 | Dimethyl ether | Examples 1 to 4 | Needle cap | |
| | 70 | 10-30 | | |
| 5 | Dimethyl ether | Examples 1 to 4 | Needle cap | |
| | 80 | 10-20 | | |

Pressing an actuator button opens a valve of the container such that pressurised propellant can force the pain relieving composition up a dip tube of the container and through the valve and cap/nozzle. The pain relieving composition is best applied as a chilled long-lasting foam.

### Example 11—Use of the Pain Relieving Aerosol Sprays

The aerosol spray of Example 9 or 10 can be used to provide topical anaesthesia of intact skin, to numb it prior to a surgical or animal husbandry procedure (eg. castration). Topical anaesthetic agents, such as lidocaine or tetracaine, usually take 20-30 minutes to work on intact skin, whereas when cooled the “cryo-anaesthetic effect” of the cooling provides rapid skin anaesthesia within seconds—allowing the skin to be surgically cut—although the cryo-anaesthetic effect wears off quickly (within a minute or two). However, then the local anaesthetic agent/s can act on the cut nerve fibres to prolong an anaesthetic effect in the open wound (eg. for castration).

In practice, the aerosol spray of Example 9 or 10 is sprayed onto a (human or animal) subject's skin or wound, usually as a long-lasting foam, for the required period of time so as to elicit instant cooling/chilling and hence a local anaesthetic effect. Although the refrigerant dissipates and the foam breaks down, the pain relieving composition forms a film over the wound and remains to provide prolonged pain relief.

The aerosol spray is particularly suitable for treating wounds caused by hot branding and hot dehorning.

The aerosol spray is particularly suitable for treating wounds caused by castration.

Advantages of the present invention as exemplified include:

The aerosol spray can be easily applied to a subject, such as an animal.

The aerosol spray can be easily applied to a large number of animals in a short period of time.

Pain relief can be provided without injection or other invasive technique.

The aerosol spray provides an initial cooling effect and almost instant pain relief.

A single application can provide both rapid and long-lasting pain relief.

The aerosol spray foam is tenacious and will stick to the wound.

The aerosol spray foam is long lasting.

Successful application of the aerosol spray is easily assessable in that a large bead of foam can be seen covering the wound.

There is very little to no run-off of the foam from the wound.

The foam breaks down to leave a pain relieving composition film over the wound.

Throughout this specification, unless in the context of usage an alternative interpretation is required, the term “comprise” (and variants thereof such as “comprising” and “comprised”) denotes the inclusion of a stated integer or integers but does not exclude the presence of another integer or other integers.

Any reference to publications cited in this specification is not an admission that the disclosures constitute common general knowledge in Australia or in other countries.

It will be appreciated by one of skill in the art that many changes can be made to the composition and uses exemplified above without departing from the broad ambit and scope of the invention.

1-20. (canceled)

21. A method selected from the group consisting of:
(a) cooling intact skin and/or a wound of a subject for pain relief and/or pain prevention;
(b) providing a subject with pain relief and/or pain prevention;
(c) providing a subject with rapid onset local anaesthesia and further pain relief; and
(d) providing a subject with rapid onset local anaesthesia immediately prior to carrying out a surgical step or animal husbandry step on the subject that produces a wound,
said method comprising the step of applying a pain relieving and pain preventing aerosol spray onto intact skin and/or wound of the subject,
wherein said aerosol spray comprises a pain relieving composition and at least one refrigerant, and
wherein the aerosol spray provides rapid onset local anaesthesia of intact skin and/or a wound of the subject to which it is applied due to the at least one refrigerant and further pain relief due to the pain relieving composition remaining on the wound of the subject.

22. The method of claim 21, wherein the aerosol spray or pain relieving composition has at least one property selected from the group consisting of:
the aerosol spray or pain relieving composition is in the form of a liquid prior to having been applied to skin or a wound;
the aerosol spray or pain relieving composition forms, or is in the form of, a sticky, viscous, adhesive gel when applied to skin or a wound;
the aerosol spray or pain relieving composition is in the form of a liquid that thickens to an adhesive gel when reacting with physiological fluids of a wound; and
the aerosol spray or pain relieving composition forms an effective long-lasting barrier over skin or a wound.

23. The method of claim 21, wherein the pain relieving composition comprises a liquid gel matrix that contains the following: at least one anaesthetic agent; a vasoconstrictor; and an antiseptic agent, and the pain relieving composition is optionally coloured.

24. The method of claim 21, wherein the pain relieving composition comprises a liquid gel matrix that contains ingredients selected from the group consisting of:
lidocaine, adrenaline and cetrimide, and the pain relieving composition is optionally coloured;
tetracaine, adrenalin and cetrimide, and the pain relieving composition is optionally coloured; and
lidocaine, bupivacaine, adrenalin and cetrimide, and the pain relieving composition is optionally coloured,
wherein said pain relieving composition has a pH lower than about 4.0.

25. The method of claim 21, wherein the pain relieving composition comprises a composition selected from the group consisting of:
(a) Composition 1 comprising:
about 100 mg/ml non-crystallising liquid sorbitol (70%);
about 50.0 mg/ml lignocaine HCl;
about 5.0 mg/ml bupivacaine HCl;
about 1.5 mg/ml sodium metabisulfite;
about 5.0 mg/ml cetrimide;
about 45.0 μg/ml adrenaline tartrate;
about 5.0 mg/ml hydroxy cellulose; and optionally
colourant;
(b) Composition 2 whereby lignocaine of Composition 1 is replaced by tetracaine at about 10 mg/ml-100 mg/ml;
(c) Composition 3 comprising:
about 100 mg/ml non-crystallising liquid sorbitol (70%);
about 40.0 mg/ml lignocaine HCl;
about 1.5 mg/ml sodium metabisulfite;
about 5.0 mg/ml cetrimide;
about 36.0 μg/ml adrenaline tartrate;
about 5.0 mg/ml hydroxy cellulose; and optionally
colourant;
(d) Composition 4 whereby lignocaine of Composition 3 is replaced by tetracaine at about 10 mg/ml-100 mg/ml;
(e) Composition 5 comprising:
about 100.0 mg/ml non-crystallising liquid sorbitol (70%);
about 50.0 mg/ml (5%) tetracaine HCl;
about 1.5 mg/ml sodium metabisulfite;
about 5.0 mg/ml cetrimide;
about 45.0 μg/ml adrenaline tartrate;
about 5.0 mg/ml hydroxy cellulose; and optionally
colourant;
(f) Composition 6 comprising: lignocaine, bupivacaine, adrenaline, cetrimide, 2-ethyl hydroxycellulose, sodium metabisulfite, liquid sorbitol (70%), buffer, and, optionally colourant;
(g) Composition 7 whereby lignocaine of Composition 6 is replaced by tetracaine at about 10 mg/ml-100 mg/ml;
(h) Composition 8 comprising: amethocaine/tetracaine, adrenaline, cetrimide, 2-ethyl hydroxycellulose, sodium metabisulfite, liquid sorbitol (70%), buffer, and, optionally, colourant;
(i) Composition 9 comprising a liquid gel matrix that contains the following: lidocaine, adrenalin, and cetrimide, and the composition is optionally coloured, wherein said composition has a pH lower than about 4.0;
(j) Composition 10 comprising a liquid gel matrix that contains the following:
tetracaine, adrenalin, and cetrimide, and the composition is optionally coloured, wherein said composition has a pH lower than about 4.0; or
(k) Composition 11 comprising a liquid gel matrix that contains the following: lidocaine, bupivacaine, adrenalin, and cetrimide, and the composition is optionally coloured, wherein said composition has a pH lower than about 4.0;
(l) Composition 12 comprising:
about 0.5% w/v cetrimide;
about 0.5% w/v hydroxyethylcellulose;
about 5% w/v tetracaine HCl;
about 0.15% w/v sodium metabisulfite;
about 0.00495% w/v adrenaline tartrate;
about 10% w/v sorbitol 70%; and optionally
dye/colourant; and
(m) Composition 13 comprising:
about 0.5% w/v cetrimide;
about 0.5% w/v hydroxyethylcellulose;
about 1% w/v tetracaine HCl;
about 0.15% w/v sodium metabisulfite;
about 0.00495% w/v adrenaline acid tartrate;
about 10% w/v sorbitol 70%; and optionally
dye/colourant.

26. The method of claim 21, wherein the at least one refrigerant comprises at least one of the following selected from the group consisting of: a compressed gas; a liquefied hydrocarbon; a fluorinated hydrocarbon; an ether; and, a hydrofluoroalkane.

27. The method of claim 21, wherein the aerosol spray is in a form selected from the group consisting of: a sprayable stream; a sprayable mist; and, a sprayable foam.

28. The method of claim 21, wherein the aerosol spray comprises or is delivered from a pressurised spray container, and comprises at least one propellant.

29. The method of claim 28, wherein the at least one refrigerant and the at least one propellant are one and the same.

30. The method of claim 28, wherein the pain relieving composition is in the form of a liquid that is expelled from the pressurised spray container as a foam and sets as a sticky viscous gel when exposed to the skin or wound, after the at least one refrigerant evaporates or otherwise dissipates.

31. The method of claim 28, wherein the pressurised spray container comprises a delivery nozzle, cap, tip or actuator.

32. The method of claim 31, wherein the delivery nozzle is in the form of a needle cap.

33. The method of claim 21, wherein the aerosol spray is applied to the subject for between about 1 and 10 seconds, and cools the skin or wound of the subject to a temperature from about −20° C. to about 10° C.

34. The method of claim 28, comprising properties selected from the group consisting of:
(a) the aerosol spray comprises a 50% w/w butane and propane blend, and 20-50% w/w pain relieving composition; the aerosol spray is delivered from a pressurised spray container comprising a delivery nozzle in the form of a needle cap; and the aerosol spray is delivered as a sticky long-lasting foam with substantially no run-off from the wound;
(b) the aerosol spray comprises a 50% w/w butane, propane and isobutane blend, and 20-50% w/w pain relieving composition; the aerosol spray is delivered from a pressurised spray container comprising a delivery nozzle in the form of a needle cap; and the aerosol spray is delivered as a sticky long-lasting foam with substantially no run-off from the wound;
(c) the aerosol spray comprises 50% w/w dimethyl ether, and 20-50% w/w pain relieving composition; the aerosol spray is delivered from a pressurised spray container comprising a delivery nozzle in the form of a needle cap; and the aerosol spray is delivered as a sticky long-lasting foam with substantially no run-off from the wound;
(d) the aerosol spray comprises 70% w/w dimethyl ether, and 10-30% w/w pain relieving composition; and, the aerosol spray is delivered from a pressurised spray container comprising a delivery nozzle in the form of a needle cap; and
(e) the aerosol spray comprises 80% w/w dimethyl ether, and 10-20% w/w pain relieving composition; and, the aerosol spray is delivered from a pressurised spray container comprising a delivery nozzle in the form of a needle cap.

35. The method of claim 21, wherein the surgical step or animal husbandry step of (d) is selected from the group consisting of: castration; mulesing; shearing; ear tagging; branding; hot branding; dehorning; hot dehorning; dis-budding; marking; treating a wound; and, treating a burn.

36. A pain relieving and pain preventing aerosol spray comprising a pain relieving composition and at least one refrigerant, wherein the aerosol spray is formulated to provide rapid onset local anaesthesia of intact skin and/or a wound of a subject to which it is applied and further pain relief due to the pain relieving composition remaining on the wound of the subject.

37. The aerosol spray of claim 36, wherein the pain relieving composition comprises a liquid gel matrix that contains ingredients selected from the group consisting of:
lidocaine, adrenaline and cetrimide, and the pain relieving composition is optionally coloured;
tetracaine, adrenalin and cetrimide, and the pain relieving composition is optionally coloured; and
lidocaine, bupivacaine, adrenalin and cetrimide, and the pain relieving composition is optionally coloured,
wherein said pain relieving composition has a pH lower than about 4.0.

38. The aerosol spray of claim 36, wherein the pain relieving composition comprises a composition selected from the group consisting of:
(a) Composition 1 comprising:
about 100 mg/ml non-crystallising liquid sorbitol (70%);
about 50.0 mg/ml lignocaine HCl;
about 5.0 mg/ml bupivacaine HCl;
about 1.5 mg/ml sodium metabisulfite;
about 5.0 mg/ml cetrimide;
about 45.0 μg/ml adrenaline tartrate;
about 5.0 mg/ml hydroxy cellulose; and optionally
colourant;
(b) Composition 2 whereby lignocaine of Composition 1 is replaced by tetracaine at about 10 mg/ml-100 mg/ml;
(c) Composition 3 comprising:
about 100 mg/ml non-crystallising liquid sorbitol (70%);
about 40.0 mg/ml lignocaine HCl;
about 1.5 mg/ml sodium metabisulfite;
about 5.0 mg/ml cetrimide;
about 36.0 μg/ml adrenaline tartrate;
about 5.0 mg/ml hydroxy cellulose; and optionally
colourant;
(d) Composition 4 whereby lignocaine of Composition 3 is replaced by tetracaine at about 10 mg/ml-100 mg/ml;
(e) Composition 5 comprising:
about 100.0 mg/ml non-crystallising liquid sorbitol (70%);
about 50.0 mg/ml (5%) tetracaine HCl;
about 1.5 mg/ml sodium metabisulfite;
about 5.0 mg/ml cetrimide;
about 45.0 m/ml adrenaline tartrate;
about 5.0 mg/ml hydroxy cellulose; and optionally
colourant;
(f) Composition 6 comprising: lignocaine, bupivacaine, adrenaline, cetrimide, 2-ethyl hydroxycellulose, sodium metabisulfite, liquid sorbitol (70%), buffer, and, optionally colourant;
(g) Composition 7 whereby lignocaine of Composition 6 is replaced by tetracaine at about 10 mg/ml-100 mg/ml;
(h) Composition 8 comprising: amethocaine/tetracaine, adrenaline, cetrimide, 2-ethyl hydroxycellulose, sodium metabisulfite, liquid sorbitol (70%), buffer, and, optionally, colourant;
(i) Composition 9 comprising a liquid gel matrix that contains the following: lidocaine; adrenalin; and cetrimide, and the composition is optionally coloured, wherein said composition has a pH lower than about 4.0;
(j) Composition 10 comprising a liquid gel matrix that contains the following:
tetracaine; adrenalin; and cetrimide, and the composition is optionally coloured, wherein said composition has a pH lower than about 4.0; or
(k) Composition 11 comprising a liquid gel matrix that contains the following: lidocaine; bupivacaine; adrenalin; and cetrimide, and the composition is optionally coloured, wherein said composition has a pH lower than about 4.0;
(l) Composition 12 comprising:
about 0.5% w/v cetrimide;
about 0.5% w/v hydroxyethylcellulose;
about 5% w/v tetracaine HCl;
about 0.15% w/v sodium metabisulfite;
about 0.00495% w/v adrenaline tartrate;
about 10% w/v sorbitol 70%; and optionally
dye/colourant; and
(m) Composition 13 comprising:
about 0.5% w/v cetrimide;
about 0.5% w/v hydroxyethylcellulose;
about 1% w/v tetracaine HCl;
about 0.15% w/v sodium metabisulfite;
about 0.00495% w/v adrenaline acid tartrate;
about 10% w/v sorbitol 70%; and optionally
dye/colourant.

39. The aerosol spray of claim 36, wherein the at least one refrigerant comprises at least one of the following ingredients selected from the group consisting of: a compressed gas; a liquefied hydrocarbon; a fluorinated hydrocarbon; an ether; and, a hydrofluoroalkane.

40. The aerosol spray of claim 36, comprising properties selected from the group consisting of:
(a) the aerosol spray comprises a 50% w/w butane and propane blend, and 20-50% w/w pain relieving composition; and, the aerosol spray forms a sticky long-lasting foam with substantially no run-off from the wound;
(b) the aerosol spray comprises a 50% w/w butane, propane and isobutane blend, and 20-50% w/w pain relieving composition; and, the aerosol spray forms a sticky long-lasting foam with substantially no run-off from the wound;
(c) the aerosol spray comprises 50% w/w dimethyl ether, and 20-50% w/w pain relieving composition; and, the aerosol spray forms a sticky long-lasting foam with substantially no run-off from the wound;
(d) the aerosol spray comprises 70% w/w dimethyl ether, and 10-30% w/w pain relieving composition; and
(e) the aerosol spray comprises 80% w/w dimethyl ether, and 10-20% w/w pain relieving composition.
